(19) United States
(12) Patent Application Publication
(10) Pub. No.: US 2006/0233888 A1
(43) Pub. Date: Oct. 19, 2006

(54) Silver coatings and methods of manufacture

(75) Inventors: Scott A. Burton, Woodbury, MN (US); David R. Holm, Hudson, WI (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/105,954

(22) Filed: Apr. 14, 2005

(51) Int. Cl. A61K 33/38; A61K 6/083

(52) U.S. Cl. 424/618; 427/2.26

(57) ABSTRACT

A silver composition comprising silver sulfate and a method of coating the composition on a substrate is disclosed.

### BACKGROUND

While wounds heal more effectively in moist environments, bacterial infection poses increased risk. Use of antibiotics to treat bacterial infections can build bacterial resistance. Silver compounds are known to impart antimicrobial effects to a surface with minimal risk of developing bacterial resistance. Silver is delivered to the surface by sustained release of silver ions from the surface when in contact with moist environments, such as a wound bed.

Silver compositions, such as silver nitrate and silver sulfadiazine, are effective antimicrobials used in a variety of applications. However, they are typically not light stable, leave a stain on skin with which they come into contact, and in the case of silver nitrate, can be quickly depleted in an aqueous environment. Use of silver salts as antimicrobials have included the use of stabilizing agents to increase light stability such as those described in U.S. Pat. No. 2,791,518 (Stokes et al.) (using a first solution of ammonia, silver nitrate and barium nitrate; and a second solution of sodium chloride and sodium sulfate); and in U.S. Pat. No. 6,669,981 (Parsons et al.) (a silver salt in water/organic solvent followed by one or more stabilizing agents (e.g., ammonium salts, thiosulphates, chlorides and/or peroxides)).

### SUMMARY

The present invention is directed to a method of coating silver sulfate on a substrate. The coated silver sulfate compositions are color stable without the addition of a stabilizing agent under readily achievable storage conditions.

In one aspect, the present invention provides a method of making an antimicrobial article, comprising preparing a composition comprising silver sulfate with the proviso that stabilizing agents are present in the composition in an amount less than 100 ppm, coating the silver sulfate composition on a substrate; and drying the coated substrate at a temperature that causes the silver sulfate composition to develop an initial color; wherein the dried silver sulfate composition is color stable.

In another aspect, an antimicrobial medical article is provided, comprising a silver sulfate composition coated on a substrate, wherein stabilizing agents are present in the antimicrobial article in an amount less than 1000 ppm and wherein the coated silver sulfate composition is color stable.

In another aspect, a method of making an antimicrobial article is provided, comprising preparing a composition of silver sulfate and water, coating the silver sulfate composition on a substrate, drying the coated substrate at a temperature that causes the silver sulfate composition to develop an initial color; and maintaining the dried silver sulfate composition at a relative humidity of no more than 50%, wherein the antimicrobial article is color stable.

In another aspect, a method of making an antimicrobial article is provided, comprising preparing a composition comprising silver sulfate, coating the silver sulfate composition on a substrate; and drying the coated substrate at a temperature that causes the silver sulfate composition to develop an initial color, wherein the dried silver sulfate composition is color stable; and wherein stabilizing agents are present in the antimicrobial article in an amount less than 1000 ppm.

In another aspect, the silver compound can be coated on a substrate such as a nonwoven gauze, a woven gauze, a film and a hydrocolloid.

“Color Stable” means that the color of the dried silver sulfate composition coated on a substrate does not exhibit a perceptible change to the human eye over time when compared to the same coated composition on a substrate that has not been exposed to light (e.g., fluorescent, natural, UV) or that has been recently. Color change can also be measured using a colorimeter according to ASTM D2244. The resulting CIELAB color difference, (DE*), between the sample after exposure for the indicated period of time and the unexposed sample can be determined. For purposes of reference only, a DE*, or color change of about 2 units is just detectable by the naked eye whereas a DE* of 20 or greater represents a substantial color change.

“Room temperature” means an average room temperature, typically 23 deg C.+/−2 deg C.

“Relative humidity” the ratio of the quantity of water vapor present in the atmosphere to the quantity that would saturate the atmosphere at the given temperature.

As used herein, “a,” “an,” “the,” “at least one,” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments.

### DETAILED DESCRIPTION OF ILLUSTRATIVE EMBODIMENTS OF THE INVENTION

The present invention provides a method for coating silver sulfate, by dissolving silver sulfate in an aqueous-based composition, coating the composition on a substrate, and drying the coated substrate. The substrate coated with silver sulfate remains stable to light (e.g., visable, UV) and heat without the addition of traditional stabilizing agents such as ammonia, ammonium salts (e.g., ammonium acetate, ammonium sulfate, and ammonium carbonate), thiosulfates, water insoluble salts of metals (e.g., halides such as chlorides), peroxides, magnesium trisilicate, and/or polymers. Preferably, any component that would function as a stabilizing agent is present in amounts less than 100 ppm, more preferably less than 50 ppm, most preferably less than 20 ppm based on the total weight of the silver sulfate composition. Alternatively, any component that would function as a stabilizing agent is present in amounts less than 1000 ppm, more preferably less than 500 ppm, most preferably less than 100 ppm based on the total weight of the antimicrobial article comprising a dried silver sulfate composition coated on a substrate.

The resultant solution containing the silver sulfate solution can be coated on a substrate, preferably an absorbent substrate, although nonabsorbent substrates can also be used. The coated substrate is dried to drive off the volatile components, such as water and organic solvents, (e.g., methanol, ethanol, isopropanol, acetone, or other organic solvents that are miscible with water). Drying can be accomplished at room temperature or by heating the coated substrate. Heat will speed the drying process. In a preferred embodiment, the coated substrate is dried at temperatures below 190° C., more preferably 170° C., even more preferably 140° C., to minimize reduction of the silver compounds, and also prevent the oxidation of a cellulosic material, when used as a substrate.

Further, tensile strength of an oxidizable substrate (such as cotton) is maximized when the silver sulfate composition on the substrate is dried at a low temperature preferably less that 140° C., more preferable at less than 100° C. and most preferably at less than 70° C.

Once dried, the substrate remains coated with the silver sulfate. The coated composition typically contains silver sulfate in a major amount. Low levels of silver metal may be present in amounts typically less than 10 wt % based on the total weight of the silver components in the composition. In some embodiments, the choice of starting materials and drying temperatures results in a coating that leaves no residue with essentially only the silver sulfate remaining on the substrate, and all other components of the silver solution removed from the substrate upon drying.

When applied, the silver sulfate solution penetrates and impregnates the interior of the substrate. For example, when gauze is used, the silver solution impregnates between the fibers of the gauze.

The concentration of silver sulfate on the substrate is a function of the amount of silver sulfate in solution, the total amount of solution applied onto a unit area of the substrate, and the drying temperature. The silver sulfate concentration on the substrate is typically less than 5 mg/cm². In a preferred embodiment, the silver sulfate concentration on the substrate ranges from 0.001 mg/cm² to 1 mg/cm².

The substrate can be a woven or nonwoven material made of natural or synthetic compounds. For example, cellulosic materials such as polysaccharide or modified polysaccharide, regenerated cellulose (such as rayon), paper, cotton, Tencel™, carboxymethyl cellulose may be used. Further, polyvinylpyrrolidone, polyvinyl alcohol, polyvinyl ether, polyacrylate, polyacrylamide, collagen, gelatin, may be used. Non-absorbent substrates may also be used including but not limited to nylon, polyester, polyethylene, and polypropylene.

The method provides a silver sulfate solution for coating on a substrate without using an acid. The presence of acid can hydrolyze the cellulosic material. This aspect of the process allows the coating to be applied without weakening the cellulosic substrate. Preferably the coating solution has a pH of at least 4, more preferably at least 5. Preferably, the coating solution has a pH no greater than 9.

Elevated temperatures can also accelerate the oxidation of cellulose by a silver salt, resulting in such affects as lowering the tensile strength and changing the color of the silver sulfate composition on the substrate. The color change on a cellulosic material such as cotton is likely due to the reduction of silver salt to silver metal with an accompanying oxidation of the cellulose substrate. The oxidized cotton has lower tensile strength.

If silver sulfate is coated on a cellulosic substate or other easily oxidizable substrate, the article will change color in proportion to the drying temperature and the time in the drying device, such as an oven. No color change is observed when the substrate coated with the silver sulfate composition is dried below approximately 100° C. for 15 minutes. For example, when wetted cotton is dried at an oven temperature greater than approximately 100° C., the cotton substrate darkens in proportion to the oven temperature and turns yellow then brown then dark brown.

If a synthetic substrate such as polyester, which is not easily oxidized, is coated with silver sulfate coating solution and dried, the polyester will remain white even when dried at temperature above 100° C.

The silver compositions, once coated, are preferably color stable. By this it is meant that the compositions are stable to at least one of the following: light, heat, and/or moisture. Regardless of substrate choice, the coated silver sulfate composition is color stable. The initial color that the silver sulfate solution develops after drying at a particular temperature, will remain without appreciable change over time either with or without exposure to light (e.g., UV, visible light).

The color stability of the coated silver sulfate composition provides a couple advantages. The color stability provides an indication to the end user that the product is of consistent high quality. Further, the color stability indicates that the form of silver on the substrate has not appreciably changed which indicates that its performance (i.e., silver release, antimicrobial activity) is essentially constant over time.

The coated compositions are stable to visible light, such that the coated compositions do not darken upon exposure to visible light. Such compositions are useful in medical articles, particularly wound dressings and wound packing materials, although a wide variety of other products can be coated with the silver sulfate compositions.

Stability of the silver sulfate coated substrate is prolonged and/or increased when the relative humidity at room temperature is maintained at 50% or lower; more preferably at 30% or lower; and most preferably at 20% or lower. Relative humidity can be reduced to 20% or lower for the silver sulfate coated substrate by a number of methods including: 1) placing the coated substrate in an environment that has a relative humidity of 20% or lower, and then packaging the product in the same environment; 2) drying the mesh in an oven, then immediately packaging the mesh; and 3) addition of a desiccant within the package. Preferably, to maintain a low relative humidity in the dried silver sulfate composition, the article should be packaged in a package with a low moisture vapor transmission rate (MVTR) such as a Techni-Pouch package (Technipaq, Inc., Crystal Lake, Ill.) with a 48 gaPET/0.00035 Foil/3.0 mil LLDPE material construction. Low relative humidity increases the thermal stability of silver sulfate treated cotton.

Silver compounds, including silver sulfate, provide sustained release of silver ions over time based in part on their limited solubility and inherent dissociation equilibrium constants. The silver sulfate composition may have other silver salts, including those that are not color stable, in varying amounts, as long as the composition when coated on the substrate remains color stable. In addition to silver sulfate, other silver compounds that may be coated on a substrate in addition to the silver sulfate include silver oxide, silver acetate, silver nitrate, silver citrate, silver chloride, silver lactate, silver phosphate, silver stearate, silver thiocyanate and silver carbonate. Preferably, the amount of silver compounds other than silver sulfate is less than 20 wt %, more preferably less than 10 wt % based on the total weight % of the silver components in the silver sulfate composition coated on the substrate.

The silver sulfate coated substrate remains stable when it contains silver sulfate in combination with other silver salts with limited color stability. Preferably, the amount of silver sulfate is at least 60 w %, more preferably at least 75 wt %, and most preferably at least 90 wt % based on the total weight % of the silver components in the silver sulfate composition coated on the substrate.

Articles can be prepared using the silver solution described herein according to a variety of coating methods. When a porous substrate is coated, the process used typically allows the yarns, filaments, or film such as perforated or microporous film, to be coated, while leaving most of the apertures unobstructed by the composition. Depending on the structure of the support used, the amount of solution employed will vary over a wide range.

The silver sulfate coating solution is prepared by mixing silver sulfate and distilled water. The silver sulfate coating solution can have a range of concentrations up to a water solubility of about 0.6% at room temperature. Optionally, higher concentrations of silver sulfate can be obtained by dissolving silver sulfate in hot water. Optionally sulfate in other forms may be added, such as sodium sulfate.

The process can be accomplished as a continuous process, or it can be done in a single step or with a single coating solution. The process to apply the coating does not require elevated temperatures, and can be applied at temperatures less than 70° C. The coating solution can be maintained below a pH of 9, and preferably less than 7, to minimize adverse effects to the substrate. The coating solution can be maintained at a pH above 4.

According to a variant of this process, a substrate can be passed through a bath of the silver composition. The substrate covered with the silver sulfate composition is then dried, for example in an oven at a temperature sufficient to evaporate constituents of the solution. The temperature is preferably less than 190° C., more preferably less than 170° C., and most preferably less than 140° C.

The silver sulfate solution can also be coated onto a carrier web or a backing (described below) using a known coating technique such as gravure coating, curtain coating, die coating, knife coating, roll coating, or spray coating. A preferred coating method is gravure coating.

Medical Articles

The silver compositions of the present invention can be used in a wide variety of products, although they are preferably used in medical articles. Such medical articles can be in the form of a wound dressing, wound packing material, or other material that is applied directly to or contacts a wound. Other potential products include clothing, bedding, masks, dust cloths, shoe inserts, diapers, and hospital materials such as blankets, surgical drapes and gowns.

The silver compositions can be coated on various backings (i.e., a support substrate). The backing or support substrate can be porous or nonporous. The composition of the present invention can be coated on the support substrate or impregnated into it, for example.

Suitable materials are preferably flexible, and may be fabric, non-woven or woven polymeric webs, polymer films, hydrocolloids, foam, metallic foils, paper, and/or combinations thereof. More specifically, cotton gauze is useful with the silver compositions of the present invention. For certain embodiments it is desirable to use a permeable (e.g., with respect to moisture vapor), open apertured substrate (i.e., a scrim). For certain embodiments, the substrate may be a hydrocolloid, such as a hydrophilic polymer, or hydrophobic polymer matrix containing hydrophilic particles, as described in applicants' copending applications, Ser. No. 10/238,577 and Ser. No. 10/728,439, both filed on Dec. 5, 2003, both of which are incorporated herein by reference.

The substrates (i.e., backings) are preferably porous to allow the passage of wound fluids, moisture vapor, and air. In certain embodiments, the substrates are substantially impervious to liquid, especially wound exudate. In certain embodiments, the substrates are capable of absorbing liquid, especially wound exudate. In certain embodiments, the substrate is an apertured liquid permeable substrate.

Suitable porous substrates include knits, wovens (e.g., cheese cloth and gauze), nonwovens (including spun-bonded nonwovens, and BMF (blown micro fibers), extruded porous sheets, and perforated sheets. The apertures (i.e., openings) in the porous substrates are of sufficient size and sufficient number to facilitate high breathability. For certain embodiments, the porous substrates have at least 1 aperture per square centimeter. For certain embodiments, the porous substrates have no greater than 225 apertures per square centimeter. For certain embodiments, the apertures have an average opening size (i.e., the largest dimension of the opening) of at least 0.1 millimeter (mm). For certain embodiments, the apertures have an average opening size (i.e., the largest dimension of the opening) of no greater than 0.5 cm.

For certain embodiments, the porous substrates have a basis weight of at least 5 grams/meter². For certain embodiments, the porous substrates have a basis weight of no greater than 200 grams/meter².

The porous substrates (i.e., backings) are preferably flexible yet resistant to tearing. For certain embodiments, the thickness of the porous substrates is at least 0.0125 mm. For certain embodiments, the thickness of the porous substrates is no greater than 3 mm.

Materials of the backing or support substrate include a wide variety of materials including paper, natural or synthetic fibers, threads and yarns made from materials such as cotton, rayon, wool, hemp, jute, nylon, polyesters, polyacetates, polyacrylics, alginates, ethylene-propylene-diene rubbers, natural rubber, polyesters, polyisobutylenes, polyolefins (e.g., polypropylene polyethylene, ethylene propylene copolymers, and ethylene butylene copolymers), polyurethanes (including polyurethane foams), vinyls including polyvinylchloride and ethylene-vinyl acetate, polyamides, polystyrenes, fiberglass, ceramic fibers, and/or combinations thereof.

The backing can also be provided with stretch-release properties. Stretch-release refers to the property of an adhesive article characterized in that, when the article is pulled from a surface, the article detaches from the surface without leaving significant visible residue. For example, a film backing can be formed from a highly extensible and highly elastic composition that includes elastomeric and thermoplastic A-B-A block copolymers, having a low rubber modulus, a lengthwise elongation to break of at least 200%, and a 50% rubber modulus of not above 2,000 pounds/square inch (13.8 megapascals (MPa)). Such backings are described in U.S. Pat. No. 4,024,312 (Korpman). Alternatively, the backing can be highly extensible and substantially non-recoverable such as those described in U.S. Pat. No. 5,516,581 (Kreckel. et al,).

In certain embodiments, the coated substrates of the present invention are nonadherent, although it should be understood that an adhesive (e.g., a pressure sensitive adhesive) could be added to an article coated with the solution. As used herein, the silver compositions of the present invention when coated on a substrate do not adhere significantly to wound tissue such that they do not cause pain and/or destruction of the wound tissue upon removal and display a 180° peel strength of less than 1 N/cm from steel, as described in applicants' copending application, Ser. No. 10/729,114, filed Dec. 5, 2003, incorporated by reference herein.

In certain embodiments, substrates coated with the silver composition can be covered on one or both sides by a permeable nonadherent outside layer to reduce adhesion and attachment to the wound. The nonadherent layer can be attached to the substrate, such as by coating or laminating. Alternatively, the coated substrate can be enclosed within a nonadherent layer, such as sleeve. The nonadherent layer can be made from nonadherent woven or nonwoven fabrics such as nylon or perflourinated-material coatings on cotton gauze. The nonadherent layer prevents attachment of materials from the enclosed silver coated substrate. At the same time, the nonadherent layer does not adversely affect the sustained release of silver from the coated substrate.

In another embodiment, the backing or support substrate can be composed of nonadherent material. For example, a nonadherent hydrophilic polymer can be used as the backing or support material, or coated on a permeable porous substrate, as described in applicants' copending applications, Ser. No. 10/728,577; Ser. No. 10/729,114; and Ser. No. 10/728,439, all of which are filed on Dec. 5, 2003.

If desired, the coated substrate can be covered with two protective films (for example, thin polyester films). These films optionally may include a nonstick treatment and can function to facilitate extraction from a package and in handling the article. If desired, the coated substrate can be cut into individual compresses, of sizes suitable for the use, packaged in sealed sachets, and sterilized.

Pressure sensitive adhesives used in medical articles can be used in articles of the present invention. That is, a pressure sensitive adhesive material could be applied to the article of this invention, for example, around the periphery, to adhere the article to the skin.

### EXAMPLES

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

GLOSSARY OF COMPONENTS

| Material/Trade | | |
|---|---|---|
| Name | Description | Source/Address |
| Ag₂SO₄ | Silver Sulfate FW311.80 | Aldrich, Milwaukee, WI |
| AgAc | Silver Acetate FW166.91 | Matheson, Coleman and |
| | | Bell, Norwood, Ohio |
| AgLac | Silver Lactate FW196.94 | Aldrich, Milwaukee, WI |
| COTTOASE | 100% cotton, spunlaced, | Unitika, Japan |
| | non-woven (<20 ppm | |
| | chloride), 50 gsm | |
| SONTARA 810 | Non-woven spunlaced | Dupont, Wilmington, DE |
| | PET, 45 gsm | |
| Spuntech | Non-woven spunlaced | Spuntech, Israel |
| | 100% cotton (700 ppm | |
| | chloride ion), 50 gsm | |
| Na₂SO₄ | Sodium Sulfate | Aldrich, Milwaukee, WI |
| AgNO₃ | Silver Nitrate | Aldrich, Milwaukee, WI |
| COLORPHAST | pH paper | EMD Chemicals, |
| | | Gibbstown, N.J. |

### Example 1

A silver sulfate coating solution was prepared by placing 0.867 g silver sulfate and 200 g distilled water in a glass bottle and capping the bottle and mixing at room temperature on a roller overnight. The pH of this solution was determined to be 5.1 using pH paper. The resulting silver sulfate (3000 μg Ag/g) solution was coated on 100% cotton spunlaced nonwoven mesh (COTTOASE, containing less than 20 ppm chloride) by transferring the solution by pipet to saturate the mesh that was contained in a polystyrene dish. The nonwoven mesh was treated with 5.5 g of the solution on a 4″ by 5″ (10.19 cm×12.7 cm) piece of mesh that weighed 0.65 g. Approximately one gram of coating solution dripped off of the mesh before the mesh was suspended in the oven for drying. Some additional solution dripped off the mesh in the oven (estimated at 1 g). The coated mesh was dried in a forced air oven (Memmert Universal Oven, available from Wisconsin Oven Company, East Troy Wis.) by heating at 66° C. for 15 minutes. The resulting Example 1 was a white appearing material. Color and color changes on exposure are noted in Table 1.

### Comparative Example A and B

A silver acetate (3000 μg Ag/g) solution was prepared and coated and dried on 100% cotton spunlaced nonwoven mesh (COTTOASE) following the procedure in Example 1 to give Comparative Example A. This silver acetate solution had a pH of 5.2 determined using pH paper. Color and color changes on exposure are noted in Table 1. A silver lactate (3000 μg Ag/g) solution was prepared and coated and dried on 100% cotton spunlaced non-woven mesh (COTTOASE) following the procedure in Example 1 to give Comparative Example B. This silver lactate solution had a pH of 5.3 determined using pH paper. Color and color changes on exposure are noted in Table 1. Samples of Examples 1 and Comparative Examples A and B were placed in a 20% constant humidity room and also in a 50% constant humidity room both at 23° C. and exposed to room fluorescent light. The color was noted visually before and after exposures and the results are presented in Table 1.

TABLE 1
Stability of Silver salts coated on Cotton

| | | | Color after | Color after |
|---|---|---|---|---|
| | | | one week at | one week at |
| | | | 20% RH under | 50% RH under |
| | Silver | Initial | fluorescent | fluorescent |
| Example | Salt | Color | light. | light |
| 1 | Silver | White | White | White |
| | sulfate | | | |
| Comparative A | Silver | White | Off white | Brown |
| | acetate | | | |
| Comparative B | Silver | White | Brown | Brown |
| | lactate | | | |

### Example 2

Non-woven spunlaced PET (Dupont SONTARA 8010; 45 gsm) was treated by coating with isopropanol. The isopropanol was removed by washing with distilled water and the resulting PET non-woven was coated with a silver sulfate solution. The silver sulfate solution was prepared as in Example 1 only sufficient Ag₂SO₄ was dissolved to make a 0.6 wt % Ag₂SO₄ solution and the sample was dried at 180° C. for 20 minutes. The resulting silver sulfate treated PET was white and did not change color after two weeks under room fluorescent lighting at room temperature and either 20% RH or 50% RH.

### Example 3

Non-woven spunlaced 100% cotton (manufactured by Spuntech, containing 700 ppm chloride ion) was coated with silver sulfate (0.6 wt % Ag₂SO₄) solution prepared as in Example 2 and dried at 60° C. for 15 minutes. Initially the material was white. It darkened in a 4 days when at 50% RH under room fluorescent lights.

### Example 4

Example 4 was prepared as in example 1 only it was dried at 140° C. for approximately 15 minutes.

### Example 5

Example 5 was prepared as in Example 1 only using a silver sulfate/sodium sulfate coating solution (3000 μg Ag/g). The coating solution was prepared as described in Example 1 with 0.34 g Na₂SO₄ added to the coating solution. This solution had a pH of 5.3 as determined using pH paper. The cotton non-woven (COTTOASE) was coated and dried at 140° C. for approximately 15 minutes.

Dried samples of Example 4 and Example 5 were placed in a bottle with a wet paper towel (100% RH), and other samples equilibrated at a given humidity (20% RH or 50% RH) and then packaged in a low MVTR heat sealable foil package to evaluate the effect of water on color stability. Color was evaluated by visual comparison with the initial sample color after one weeks aging.

TABLE 2
Color of treated Cotton on Aging

| | | Color After One Weeks Aging | |
|---|---|---|---|
| | | at Conditions | |
| | Initial Color | Example 4 | Example 5 |
| | Example 4 & | Ag₂SO₄ treated | Ag₂SO₄/Na₂SO₄ |
| Conditions | Example 5 | cotton | treated cotton |
| 22° C.; 20% RH | pale yellow | pale yellow | pale yellow |
| 22° C.; 100% RH; | pale yellow | mottled yellow | mottled yellow |
| 49° C.; 20% RH | pale yellow | pale yellow | pale yellow |
| 49° C.: 50% RH | pale yellow | pale yellow | pale yellow |
| 49° C.; 100% RH | pale yellow | mottled brown | mottled brown |
| 49° C.; 100% RH | pale yellow | brown | brown |

### Example 6

Example 6 was made by using the procedure outlined in Example 1 only the coating solution contained a mixture of silver nitrate and silver sulfate. The coating solution was made by mixing 90 g of silver sulfate aqueous solution (3000 μg Ag/g) and 10 g of silver nitrate aqueous solution (3000 μg Ag/g). This solution had a pH of 5.3 determined using pH paper. The cotton non-woven (COTTOASE) was coated with this silver sulfate/silver nitrate coating solution and then dried at 60° C. for 15 minutes. The dried mesh was white and was color stable (under room fluorescent lighting and room temperature) at 20% RH and 50% RH for more than 24 hours.

### Examples 7-10

Examples 7-10 were made as in Example 1. A silver sulfate coating solution was made by mixing 0.867 g silver sulfate and 200 g distilled water. Cotton non-woven (COTTOASE) was coated with this solution and dried at the temperatures in Table 3 to make Examples 7-10.

### Examples 11-14

Examples 11-14 were made as in Example 1 only a silver sulfate/sodium sulfate coating solution (3000 μg Ag/g) was used. The solution was made by mixing 0.867 g silver sulfate, 0.34 g Na₂SO₄, and 200 g distilled water. Cotton non-woven (COTTOASE) was coated with this solution and dried at the temperatures in Table 3 to make Examples 11-14.

Effect of drying temperature on cotton non-woven (COTTOASE) tensile strength (cross direction) and color were determined where the cotton was treated with water (Control), silver sulfate solution (3000 μg Ag/g, Examples 7-10), or silver sulfate/sodium sulfate solution (3000 μg Ag/g, Example 11-14) and dried in an oven for 15 minutes at the indicated temperatures in Table 3. Tensile Strength was determined according to ASTM Test Method No. D3759-83 and was performed using a Thwing Albert tester (Model EJA/2000, Thwing Albert Company, Philadelphia, Pa.), a sample width of 2.54 cm, a gauge length of 10.16 cm, and a crosshead speed of 12.7 cm/min. Reported is the maximum force applied to the test sample to obtain the tensile value at point of break divided by sample width. The units are force (N) per unit of sample width (cm). Tensile strength at break was run on 9 cross direction samples for each Example determination. The results in the table are the mean with standard deviations reported in parentheses.

TABLE 3
Effect of Drying Temperature on Tensile

| | Water | | | | | Silver sulfate + | | |
|---|---|---|---|---|---|---|---|---|
| Oven | Control | | Silver sulfate | | | sodium sulfate | | |
| Temp | Tensile | | Example | Tensile | | Example | Tensile | |
| (° C.) | (N/cm) | color | No. | (N/cm) | color | No. | (N/cm) | color |
| 60 | 3.77 | white | 7 | 4.13 | white | 11 | 4.17 | white |
| | (0.77) | | | (0.63) | | | (0.46) | |
| 140 | 3.70 | white | 8 | 3.42 | tan | 12 | 3.92 | tan |
| | (0.53) | | | (0.44) | | | (0.70) | |
| 160 | nr | nr | 9 | 2.14 | yellow | 13 | 2.59 | yellow |
| | | | | (0.19) | | | (0.49) | |
| 190 | 3.49 | white | 10 | 0.28 | brown | 14 | 0.63 | brown |
| | (0.61) | | | (0.04) | | | (0.09) | |

Tensile-Mean (SD); n = 9; the untreated control tensile was 3.87 (0.44) N/cm.
nr—sample not run

High drying temperature affects the tensile strength and color of the silver mesh. The addition of sodium sulfate lessened the tensile strength degradation.

### Example 15

Example 15 was prepared as in Example 1 using cotton non-woven (COTTOASE) coated with silver sulfate (0.6 wt % Ag₂SO₄) solution and dried at 125° C. for approximately 25 minutes. The resulting color was light yellow. The silver treated cotton was exposed to fluorescent light for two weeks at approximately 20% RH with no color change. Silver ion release was measured using a silver ion selective electrode (Orion, available VWR International, Batavia, Ill.). Silver release of 30 mg silver ion per gram Example 15 was measured within two minutes of placing the sample in distilled water.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method of making an antimicrobial article, comprising:
Preparing a composition comprising silver sulfate with the proviso that stabilizing agents are present in the composition in an amount less than 100 ppm;
Coating the silver sulfate composition on a substrate; and
Drying the coated substrate at a temperature that causes the silver sulfate composition to develop an initial color;
Wherein the dried silver sulfate composition is color stable.

2. The method of claim 1, wherein the stabilizing agents are present in amounts less than 50 ppm.

3. The method of claim 1, wherein the stabilizing agents are present in amounts less than 20 ppm.

4. The method of claim 1, wherein the antimicrobial article is maintained in an environment of no more than 50% RH at room temperature.

5. The method of claim 1, wherein the antimicrobial article is maintained in an environment of no more than 20% RH at room temperature.

6. The method of claim 1, wherein the silver sulfate composition further comprises silver compounds other than silver sulfate.

7. The method of claim 6 wherein the silver compounds are selected from the group consisting of silver oxide, silver nitrate, silver acetate, silver citrate, silver chloride, silver lactate, silver phosphate, silver stearate, silver thiocyanate and silver carbonate.

8. The method of claim 6 wherein the silver compounds comprise less than 20 wt % of the composition based on the total weight of the silver components of the composition.

9. The method of claim 1, wherein the silver sulfate is present in an amount of at least 60 wt % of the composition based on the total weight of the silver components of the composition.

10. The method of claim 9 wherein the silver sulfate is present in an amount of at least 90 wt % of the composition based on the total weight of the silver components of the composition.

11. The method of claim 1, wherein the pH of the coating solution is at least 4.

12. The method of claim 1, wherein the pH of the coating solution is no greater than 9.

13. The method of claim 1, wherein the silver sulfate composition is essentially free of any acid.

14. The method of claim 1, wherein the initial color is selected from the group consisting of white, yellow, gold, tan, and brown.

15. The method of claim 1, wherein the temperature is less than 190 deg C.

16. The method of claim 1, wherein the temperature is less than 170 deg C.

17. The method of claim 1, wherein the temperature is less than 140 deg C.

18. The method of claim 1, wherein the temperature is less 70 deg C.

19. The method of claim 1, wherein the substrate is a nonwoven gauze, a woven gauze, a film or a hydrocolloid.

20. The method of claim 19, wherein the substrate comprises a material selected from the group consisting of a cellulosic material and a polyester fiber.

21. An antimicrobial medical article comprising a silver sulfate composition coated on a substrate, wherein stabilizing agents are present in the antimicrobial article in an amount less than 1000 ppm and wherein the coated silver sulfate composition is color stable.

22. The article of claim 21, wherein the stabilizing agents are present in amounts less than 500 ppm.

23. The article of claim 21, wherein the stabilizing agents are present in amounts less than 100 ppm.

24. The article of claim 21, wherein the antimicrobial article is maintained in an environment of no more than 50% RH at room temperature.

25. The article of claim 21, wherein the antimicrobial article is maintained in an environment of no more than 20% RH at room temperature.

26. The article of claim 21, wherein the silver sulfate composition further comprises silver compounds other than silver sulfate.

27. The article of claim 26, wherein the silver compounds are selected from the group consisting of silver oxide, silver nitrate, silver acetate, silver chloride, silver lactate, silver phosphate, silver stearate, silver thiocyanate and silver carbonate.

28. The article of claim 26, wherein the silver compounds comprise less than 20 wt % of the composition based on the total weight of the silver components of the composition.

29. The article of claim 26, wherein the silver sulfate is present in the composition in an amount of at least 90 wt % of the composition based on the total weight of the silver components of the composition.

30. The article of claim 26, wherein the silver sulfate composition has an initial color selected from the group consisting of white, yellow, gold, tan, and brown.

31. The article of claim 26, wherein the substrate is a nonwoven gauze, a woven gauze, a film or a hydrocolloid.

32. The article of claim 31, wherein the substrate comprises a material selected from the group consisting of a cellulosic material, nylon, and a polyester fiber.

33. A method of making an antimicrobial article, comprising:
Preparing a composition of silver sulfate and water;
Coating the silver sulfate composition on a substrate;
Drying the coated substrate at a temperature that causes the silver sulfate composition to develop an initial color; and
Maintaining the dried silver sulfate composition at a relative humidity of no more than 50%;
Wherein the antimicrobial article is color stable.

34. A method of making an antimicrobial article, comprising
Preparing a composition comprising silver sulfate;
Coating the silver sulfate composition on a substrate; and
Drying the coated substrate at a temperature that causes the silver sulfate composition to develop an initial color;
Wherein the dried silver sulfate composition is color stable; and
Wherein stabilizing agents are present in the antimicrobial article in an amount less than 1000 ppm.

35. The method of claim 1, wherein the stabilizing agents are present in amounts less than 500 ppm.

36. The method of claim 1, wherein the stabilizing agents are present in amounts less than 100 ppm.
